(19) United States
(12) Patent Application Publication
(10) Pub. No.: US 2012/0265166 A1
(43) Pub. Date: Oct. 18, 2012

(54) DEVICES AND METHODS FOR ENHANCING DRUG ABSORPTION RATE

(76) Inventor: Ofer Yodfat, Modi'in (IL)

(21) Appl. No.: 13/260,820

(22) PCT Filed: Apr. 6, 2010
(86) PCT No.: PCT/IL2010/000275

§ 371 Date: Dec. 8, 2011

Related U.S. Application Data
(60) Provisional application No. 61/164,787, filed on Mar. 30, 2009.

(51) Int. Cl. A61M 5/158; A61M 5/168; A61M 5/142

(52) U.S. Cl. 604/506; 604/151; 604/272

(57) ABSTRACT

Devices, systems and methods directed to a drug delivery device including a soft subcutaneously insertable cannula are disclosed. Some embodiments of the cannula include an elongated soft tube having a plurality of apertures spaced around and/or along a wall of the elongated soft tube. The plurality of apertures is configured for fluid flow therethrough resulting-in/causing an increase in an absorption rate of the fluid in the body of the user. The drug delivery device can be an insulin pump.

### CROSS-REFERENCE TO RELATED APPLICATIONS

This application claims priority to U.S. provisional application No. 61/164,787, entitled “Devices and Methods for Enhancing Drug Absorption Rate” filed Mar. 30, 2009, the content of which is hereby incorporated by reference in its entirety.

### FIELD

Devices, systems and methods for enhancing absorption rate of drugs in a tissue are described herein. In particular, some embodiments disclosed herein include an ambulatory portable infusion device that can be attached to the user's/patient's body and dispense doses of fluids to the patient's body. More particularly, some embodiments of the present disclosure are directed to a skin adherable infusion device that includes a soft cannula provided with a plurality of openings/holes spaced apart from one another around and along the cannula, to dispense fluid to the patient's subcutaneous tissue. The disclosure also includes embodiments directed to a method for improving fluid delivery absorption into the patient's subcutaneous tissue, and thus, to the systemic circulation is described herein. The terms “fluid” and “drug” refer to any therapeutic fluid, including but not limited to insulin.

### BACKGROUND

Tight glycemic control is essential in patients who require insulin for the treatment of diabetes, and its benefits have been demonstrated in several prospective clinical trials such as in the Diabetes Control and Complications Trial (DCCT) and in the U.K. Prospective Diabetes Study (UKPDS), (N Engl J Med 329:977-986, 1993, Lancet 352:854-865, 1998). Regimens involving multiple daily injections (“MDI”) of insulin and/or continuous subcutaneous insulin injection (“CSII” or using insulin pumps) are designed to achieve tight glycemic control, attempting to mimic physiologic insulin secretion. The normal pancreas regulates insulin secretion to counteract alterations (i.e., elevations or drops) in blood glucose levels and maintain substantially constant glucose levels regardless diet or daily activity, i.e., regulating insulin and glucose in a closed loop mode. A complex array of physiological events occurs prior to eating (often referred-to as the cephalic phase of insulin secretion) which prepares the pancreas for immediate release of preformed insulin when blood glucose levels increase in response to food intake. This immediate insulin secretion prepares the tissues (primarily muscle and liver) to rapidly take up glucose molecules and thereby prevent severe postprandial hyperglycemia.

In type 1 diabetes patients, very little, if any, endogenous insulin is available to handle (i.e., counteract) the carbohydrate load which rapidly enters the circulation as eating begins. Before the advent of rapid-acting insulin analogues (e.g., Lispro, Aspart, Glulisine), regular human insulin (“RHI”) had been a preferable treatment to address postprandial hyperglycemia. However, RHI demonstrated a delayed onset of activity after subcutaneous administration (peaks at 90-120 minutes after injection), resulting in a recommendation that it should be injected at least 30 min before a meal (American Diabetes Association: Clinical practice recommendations 2003: insulin administration. Diabetes Care 26 (Suppl. 1):S121-S124, 2003). Adherence to this recommendation may be inconvenient and has resulted in many patients who negligently injected RHI closer than 30 min to a meal.

The absorption of insulin analogues from the subcutaneous tissue into the blood tissue is faster than that of RHI but does not occur for the first 15 minutes following injection, and blood insulin levels peak at 40-60 minutes after injection. Typically, no postprandial excursion is observed when insulin is administered 30 minutes prior to the meal, but when insulin is administered right at mealtime (which is the most common pattern among type 1 diabetic patients), severe postprandial hyperglycemia (hereinafter “PPH”) can be observed. Based on studies, there is some evidence to suggest that in diabetic patients, PPH adds more to the total hyperglycemic burden and associated cardiovascular risk. More recent evidence suggests that, in addition to an existing risk of chronic hyperglycemia, excessive postprandial excursions may provide additional risks for the development of cardiovascular diseases. Conventional means for increasing insulin absorption rate at the subcutaneous tissue are aimed towards improvement of the pharmacokinetic and pharmacodynamic properties of insulin formulations.

After injection of insulin into the subcutaneous tissue, it is absorbed from the insulin depot into the blood complying with a first order kinetic behavior.

Insulin molecules from the depot diffuse through a surface in a first order process according to the following equation:

dX/dT=λX
wherein:
dX/dT—insulin exit rate expressed in mass per time unit
X—insulin mass contained in the depot
λ—first order rate constant
   From this equation the following equation can be obtained:

λ=(dX/dT)/X=(dX/X)/dT

This equation means fraction of the molecules transfer outside the insulin depot per infinitesimal of time. The largest value of λ, dX=X, is obtained when all molecules are instantaneously transferred. This situation will happen when, in the absence of any thermodynamic impediment, all the molecules are contained in a depot that does not allow them to move in another direction and in another trajectory length other than exit out of the depot. Ideally, this would be a depot with all insulin molecules are attached to the surface at minimum perpendicular distance. If the surface area is increased without modifying the distance to it, no modification in λ would be observed. Both the surface area and the volume of the depot would be increased in the same proportion. If the distance to the surface is increased leaving the area unchanged, the instantaneously transferred amount of insulin will necessary decreases, as dX<X. This reasoning leads to assess an inverse relationship between λ and the perpendicular distance to the transfer surface from the opposite edge of the depot. In other words, λ is related in direct proportion to the surface area of the depot (A) and inverse proportion to the depot volume (V).

If the depot volume (assuming a perfect spherical shape) V=4πR³/3 and the surface area is A=4πR² the surface/volume ratio A/V=3/R. The absorption rate is maximal when the depot radius is getting smaller (infinitesimal). A simple way to increase A/V ratio (reducing R) is to divide the insulin depot to a number (n) of depots. For example a single depot of 10 U insulin with a concentration of 100 U/ml (V=100 mm³) has a surface area of ˜104 mm² and 10 depots (n=10) of 1 U (V=10×10 mm³=100 mm³) have in total a surface area of ˜224 mm² (more than double).

The most common way to administer insulin is by a syringe having a sharp needle (typically made out of metal). The insulin is injected subcutaneously at one site creating a single depot that is gradually absorbed to the bloodstream. An improvement for this method using a “sprinkler needle” has been described by Berit Edsberg et al (BMJ 1987; 294: 1373-6), who employed a 25 gauge metal needle containing 14 holes in its walls and sealed at its tip. Insulin was absorbed more rapidly and glucose levels raised less after insulin injection using the sprinkler needle versus a “normal” non-porous needle. U.S. Pat. Nos. 4,413,993 (Guttman), 4,790,830 (Hamacher), and 4,838,877 (Massau), each disclose a hypodermic or intravenous delivery needle having one or more apertures located on the side of a sharpened tip needle. U.S. Pat. No. 2,748,769 (Huber) discloses a hypodermic needle having a curved or bent tip cut in a plane that extends along the side of the needle towards which the bend is made and thereby providing an orifice which is not plugged by tissue upon insertion into a subject, the curved surface being provided with an auxiliary delivery orifice which ensures delivery when the main orifice rests against a vein wall. U.S. Pat. No. 3,076,457 (Copen) discloses a hypodermic needle having an aperture at the tip and also having an opening which extends along the side of the shaft for part of its length. U.S. Pat. No. 6,261,272 (Gross) discloses a metal needle having pores in its walls and a sharp tip. The porous needle can be connected to a fluid delivery device. Gross mainly relates to the production process of drilling a cut extending across the external surface of the side of the needle shaft and the external aperture area is greater than the internal area. Another “infiltration cannula” is described in U.S. patent application published as 2007/0106234. A metal porous needle is connected to a hub which is held by the operator during administration of drug into the subcutaneous tissue. In the above mentioned patents and patent application, the needles that penetrate the skin are also serving as infiltrating means (sprinkler needle). Needles made of metal or other rigid materials suffer from significant drawbacks, particularly when long term insertion is required (e.g., during several days) because they cause constant pricking sensation and continuous micro-traumas during body movements. These limitations are further augmented in diabetic patients using insulin pumps who need continuous insulin administration around the clock.

Insulin pumps (or CSII) deliver rapid acting insulin 24 hours a day through a cannula placed under the skin. The insulin total daily dose (“TDD”) is divided into basal and bolus doses. Basal insulin is delivered continuously over 24 hours, and keeps the blood glucose levels in range (i.e., euglycemia) between meals and overnight. Diurnal basal rates can be pre-programmed or manually changed according to various daily activities. Insulin boluses are typically delivered before or during meals to counteract carbohydrates loads or during episodes of high blood sugar levels. Conventional insulin pumps include two types of pumps: a portable pager-like device that delivers insulin via a long tubing infusion set (hereinafter “pager pump”) and a skin adherable tubeless dispensing patch (hereinafter “patch pump”).

Comparative studies have shown that clinical outcome (i.e., HbA1c reduction) of pump users over MDI is negligible (Diabetes Care 2008; 31(Supp. 2): S140-145). This poor outcome may stem in CSII inability to mitigate PPH. Most pump users administer bolus at mealtime (i.e., upon food intake) and the rapid rise of blood glucose levels cannot be counteracted because the absorption of rapid acting insulin from the subcutaneous tissue lags behind glucose absorption from the gut.

Co-owned U.S. patent application Ser. Nos. 11/397,115, 12/004,837, and International Patent Application Nos. PCT/IL09/000,388 (claiming priority to U.S. Provisional Application No. 61/123,509) and PCT/IL08/001,057, disclose a skin adherable insulin dispensing pump that can deliver insulin into the subcutaneous tissue through a soft cannula, the disclosures of which are incorporated herein by reference in their entireties.

In co-owned U.S. patent application Ser. Nos. 11/706,606, 11/963,481 and International Patent Application No. PCT/IL08/001,521, the disclosures of which are incorporated herein by reference in their entireties, a device that contains means for both insulin dispensing and glucose sensing using a single cannula (or probe) is disclosed. In both the “stand alone” dispensing device and dispensing/sensing device, the soft cannula is inserted to the subcutaneous tissue using a sharp metal penetrating member that is retracted after insertion as further described in co-owned U.S. patent application Ser. Nos. 11/989,684, 12/004,837, 12/215,219 and 12/215,255, the disclosures of which are incorporated herein by reference in their entireties. This cannula has a single opening at the distal end, and thus a single insulin depot is formed upon each bolus or basal dose administration.

### SUMMARY

The present disclosure describes embodiments which address the shortcomings noted with current and past devices.

Accordingly, in some embodiments, devices, systems and methods are provided which provide a drug (e.g., insulin) infusion device and a method for accelerating and/or enhancing the drug absorption in tissue. This acceleration can be implemented by increasing the depot's surface to volume (A/V) ratio in the tissue (e.g., subcutaneous, intradermal, cutaneous).

In some embodiments disclosed herein, a device that delivers insulin into the body and can concomitantly monitor body glucose (e.g., blood glucose, ISF glucose) levels is provided, as well as a method for accelerating insulin absorption. This acceleration can be implemented by increasing the tissue depot's surface to volume (A/V) ratio.

In some embodiments disclosed herein, a device which is miniature, discreet, economical for users/patients and highly cost effective is provided, as well as a method for accelerating insulin absorption by increasing the subcutaneous depot's surface to volume (A/V) ratio.

In some embodiments disclosed herein, a device that contains a miniature skin securable dispensing patch unit that can continuously dispense insulin is provided as well as a method for accelerating insulin absorption by increasing the subcutaneous depot's surface to volume (A/V) ratio.

In some embodiments disclosed herein, a device that comprises insulin dispensing patch unit that can be remotely controlled is provided and a method for accelerating insulin absorption by increasing the subcutaneous depot's surface to volume (A/V) ratio.

In some embodiments, a miniature skin securable patch is provided that can continuously dispense insulin and monitor body glucose concentration levels and a method accelerating insulin absorption by increasing the subcutaneous depot's surface to volume (A/V) ratio.

In some embodiments, a miniature skin securable patch is provided that can continuously dispense insulin and continuously monitor body glucose concentration levels and a method accelerating insulin absorption by increasing the subcutaneous depot's surface to volume (A/V) ratio.

In some embodiments, a device is provided that includes a closed or semi-closed loop system that is capable of monitoring glucose levels and dispensing insulin according to the sensed glucose levels and a method for accelerating insulin absorption by increasing the subcutaneous depot's surface to volume (A/V) ratio.

Some embodiments of the present disclosure are directed to a drug delivery device for dispensing of a drug or other therapeutic fluid into a body of a user/patient. The device may include a reservoir retaining a drug, a cannula insertable into a tissue of the body of a user, and a pump for dispensing the drug from the reservoir into the tissue via the cannula. The cannula may comprise an elongated tube having a plurality of apertures spaced around and/or along a wall of the elongated tube (hereinafter a “soft sprinkler cannula” or a “sprinkler cannula” or a “sprinkler”). The plurality of apertures is configured for delivering the drug into the tissue. The tube may be soft/flexible. The soft tube can be made from a polymer (e.g., Teflon®).

In some embodiments, the plurality of apertures forms/corresponds to a plurality of depots for the fluid. The plurality of apertures causes/results in an increase in absorption rate of the fluid in the tissue, and thus, in the body of the user/patient. The plurality of depots may further include an effective diffusion area substantially larger than an effective diffusion area formed from a single aperture forming a single depot.

In some embodiments, the cannula of the device can be provided with a penetrating member having a sharp tip. The penetrating member is capable of longitudinally traversing through the cannula. After cannula insertion into the body, the penetrating member can be retracted and the cannula is being retained within the tissue. The cannula may be retained within the tissue, e.g., for 2 to 7 days, or preferably for about 3 days.

The device, according to some embodiments of the present disclosure, includes a dispensing unit (or a dispensing patch unit), and in some embodiments, the device may further include a remote control unit (also referred-to as “remote control” or “RC”). Such an RC may be capable of communicating with the dispensing unit and may enable at least one of: programming of therapeutic fluid delivery, receiving user input, and data acquisition. The dispensing unit may comprise a pump. In some embodiments, the pump can include a syringe with a movable plunger. In alternative embodiments, the pump may include a peristaltic pump including a rotatable member configured for squeezing a delivery tube. The dispensing unit can be connected to a tissue (e.g., subcutaneous) insertable cannula through which drug (e.g., insulin) is delivered to the body of a user/patient. In some embodiments, the dispensing unit can be comprised of two parts: a disposable part (“DP”) and a reusable part (“RP”). In some embodiments, the DP may include at least the reservoir, and the RP may include at least a portion of the pump. In some embodiments, the DP can include a disposable part housing and the RP can include a reusable part housing. Upon connection of the two parts or housings, the dispensing unit becomes operable, enabling drug flow from the reservoir to the tissue/body of the patient. In some embodiments, a cradle unit (also referred-to as “cradle”) is provided, which enables dispensing unit disconnection and reconnection upon patient's discretion. In some embodiments, the cradle can be a flat sheet that adheres to the skin. After attachment of the cradle unit to the skin, a sprinkler cannula can be inserted into a tissue compartment (e.g., subcutaneous) through a dedicated passageway in the cradle unit. The sprinkler cannula can be inserted manually or automatically using a designated inserter device at various insertion angles.

In some embodiments, a cannula for dispensing of a fluid to a body of a user/patient, the cannula is provided and comprises one or more of an elongated tube having a plurality of apertures spaced apart around and/or along a wall of the elongated tube. The plurality of apertures is configured for delivering a drug into the tissue of a user/patient. A connector may also be provided on a proximal end of the tube for establishing fluid communication between a fluid delivery device and the cannula, where the cannula is insertable into tissue by using a rigid penetrating member having a sharp tip. The plurality of apertures results-in/causes an increase in an absorption rate of the fluid in the body of the user/patient.

In some embodiments, the plurality of apertures forms/corresponds to a plurality of depots for the fluid, and the plurality of depots include an effective diffusion area substantially larger than an effective diffusion area formed from a single aperture forming a single depot.

The plurality of apertures may be configured with substantially similar dimensions. In addition, in some embodiments, at least one aperture of the plurality of apertures is configured with one or more dimensions different from another aperture of the plurality of apertures. In some embodiments, the cannula comprises a tip, and the tip is provided with an opening which may include a self-sealable septum.

In some embodiments, one or more of the plurality of apertures of the cannula include at least one unidirectional valve.

In some embodiments, a method for increasing absorption rate of a therapeutic fluid into a tissue of the body of a user/patient is provided and may include one or more of the following steps: providing a cannula for dispensing of the therapeutic fluid into a tissue of the body of a patient, where the cannula includes an elongated soft tube having a plurality of apertures disposed around and/or along a wall of the elongated soft tube, the plurality of apertures being configured for delivering therapeutic fluid (e.g., a drug) into the tissue of the user/patient, and a connector may be provided on a proximal end of the elongated soft tube for establishing fluid communication between a fluid delivery device and the cannula. The method may also include inserting the cannula into a patient's tissue via a rigid penetrating member having a sharp tip and dispensing the therapeutic fluid through the cannula. The therapeutic fluid flows through the plurality of apertures resulting-in/causing an increase in the absorption rate of the therapeutic fluid in the tissue/body of the patient.

Some embodiments of the present disclosure are directed to a soft cannula having a plurality of pores/openings/apertures in its side walls (hereinafter a “soft sprinkler cannula” or a “sprinkler cannula” or a “sprinkler”) such that upon drug administration a plurality of depots are formed. Such pores are preferably spaced apart from one another around an along the side walls of the cannula.

In some embodiments, at least one pore is located so that at least one depot is formed in the cutaneous tissue.

In some embodiments, the absorption rate of insulin is influenced by the circulation of blood in the vicinity of the injection site, and insulin absorption (for example) at the injection site is enhanced with such increased blood flow. Since cutaneous tissue is more vascular than subcutaneous tissue compartments, insulin delivered according to some embodiments to the cutaneous tissue, absorbs more rapidly than insulin delivered subcutaneously. Such insulin delivery is via at least some embodiments of the present disclosure which include a sprinkler cannula positioned in an intradermal tissue, for example. An associated method according to some embodiments enhances therapeutic fluid absorption from the injection site into the systemic circulation.

Other embodiments of the present disclosure include one or more, and various combinations of the elements and features noted above, as well as in combination with other elements and features set out below.

### BRIEF DESCRIPTION OF DRAWINGS

FIGS. 1a-b illustrate an infusion device with a vertical (FIG. 1a) and skewed (FIG. 1b) subcutaneously inserted sprinkler cannula according to some embodiments of the disclosure.

FIG. 2 illustrates a pager pump with an infusion set and a sprinkler cannula according to some embodiments of the disclosure.

FIG. 3 illustrates a skin adherable port with a sprinkler cannula and a syringe for fluid administration via the cannula according to some embodiments of the disclosure.

FIGS. 4a-b illustrate a two-part pump adherable directly (FIG. 4a) and via a cradle unit (FIG. 4b) to the patient's skin according to some embodiments of the disclosure.

FIGS. 5a-c illustrate cross sectional views of: a skin adherable cradle (FIG. 5a) and a sprinkler cannula before (FIG. 5b) and after (FIG. 5c) insertion through a cradle opening according to some embodiments of the disclosure.

FIGS. 6a-b illustrate spatial views of a cradle after horizontal (FIG. 6a) and skewed (FIG. 6b) sprinkler cannula insertion according to some embodiments of the disclosure.

FIG. 7 illustrates an infusion device that is composed of a skin adherable cradle, a dispensing unit that can be disconnected from the cradle, and a remote control unit that contains a blood glucose monitor according to some embodiments of the disclosure.

FIG. 8 illustrates a dispensing unit that is composed of a reusable part and a disposable part according to some embodiments of the disclosure.

FIGS. 9a-c illustrate a spatial view of a cradle (FIG. 9a), and a normal (FIG. 9b) and magnified (FIG. 9c) bottom views of a sprinkler cannula according to some embodiments of the disclosure.

FIGS. 10a-b illustrate a cross sectional normal (FIG. 10a) and magnified (FIG. 10b) views of a cradle and a skewed sprinkler cannula according to some embodiments of the disclosure.

FIGS. 11a-b illustrate a spatial view of skewed sprinkler cannula before (FIG. 11a) and after (FIG. 11b) insertion through an opening within a cradle according to some embodiments of the disclosure.

FIGS. 12a-b illustrate a normal (FIG. 12a) and magnified (FIG. 12b) views of a sprinkler cannula having a self sealed tip according to some embodiments of the disclosure.

FIG. 13 illustrates a two-part dispensing unit connected to a skin adherable cradle and a skewed sprinkler cannula according to some embodiments of the disclosure.

FIGS. 14a-b illustrate cross sectional views of a two-part dispensing unit before (FIG. 14a) and after (FIG. 14b) connection to a skin adherable cradle and a skewed sprinkler cannula according to some embodiments of the disclosure.

FIGS. 15a-c illustrate cross sectional views of fluid (e.g., insulin) depots emerging from a subcutaneous cannula. FIG. 15a illustrates one depot emerging from a cannula. FIG. 15b illustrates a vertical sprinkler cannula and drug depots emerging from the cannula holes. FIG. 15c illustrates a skewed sprinkler cannula and drug depots emerging from the cannula holes according to some embodiments of the disclosure.

FIG. 16 illustrates a magnified view of a sprinkler cannula having holes and comprising unidirectional valves according to some embodiments of the disclosure.

FIGS. 17a-c illustrate a dispensing unit (or patch unit) that contains a dispensing apparatus and a sensing apparatus according to some embodiments of the disclosure. The patch unit is connected to a cradle and a sprinkler cannula that includes a sensing element. FIG. 17a illustrates a cross sectional view of the patch, cradle and sprinkler cannula with the sensing element. FIGS. 17b (normal view) and 17c (magnified view) illustrate a spatial views of the sprinkler cannula and the sensing element.

### DETAILED DESCRIPTION

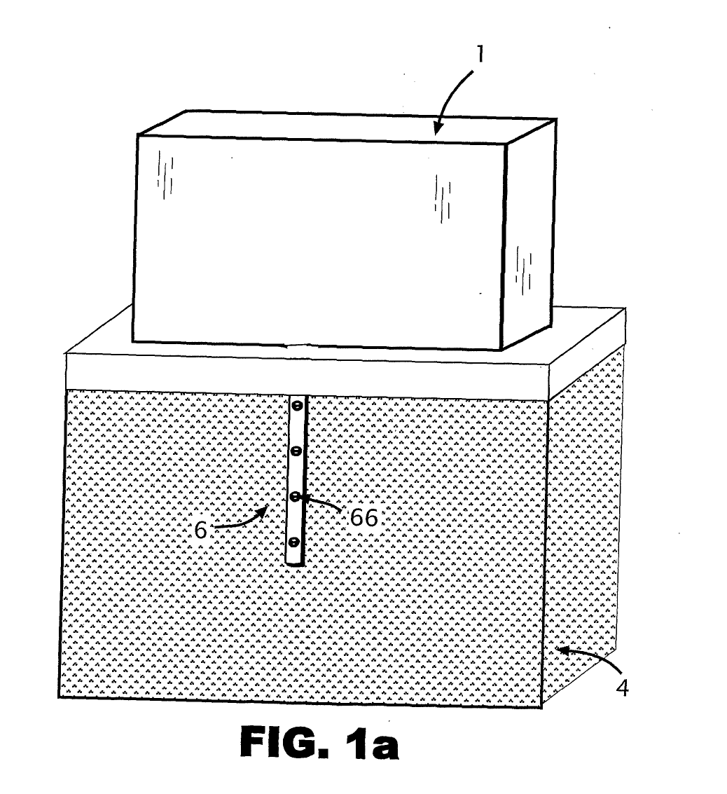

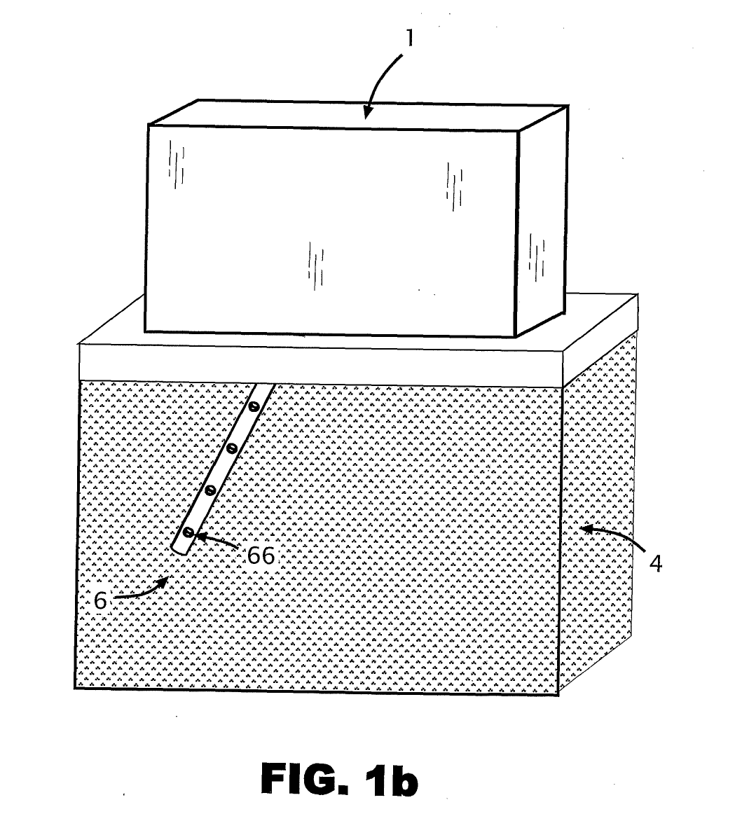

FIGS. 1a-b illustrate embodiments of an infusion device 1 that can deliver therapeutic fluid (e.g., insulin) into the body of a patient through a soft cannula 6 that includes holes 66 along its longitudinal axis (hereinafter a “sprinkler cannula”). Therapeutic fluid(s) can be delivered to several tissues such as the cutaneous tissue, subcutaneous compartment 4, soft tissues (e.g., muscles), or blood vessels (e.g., veins or arteries). The sprinkler cannula 6 can be inserted into the subcutaneous tissue 4 in various angles with respect to the skin surface, e.g., perpendicularly (or vertically) (see FIG. 1a), horizontally or in skewed/tilted manner (see FIG. 1b). The sprinkler cannula may be provided with a plurality of holes to enable higher drug absorption rates in the tissue by increasing the total surface area of the drug depots emerging from the sprinkler cannula holes. In other words, a single volume of drug (i.e., a dose) can be divided to several depots, thus, the total surface area of all depots is higher than that of the single depot delivered from a conventional cannula which has a single opening (typically at the cannula tip) to allow drug exit.

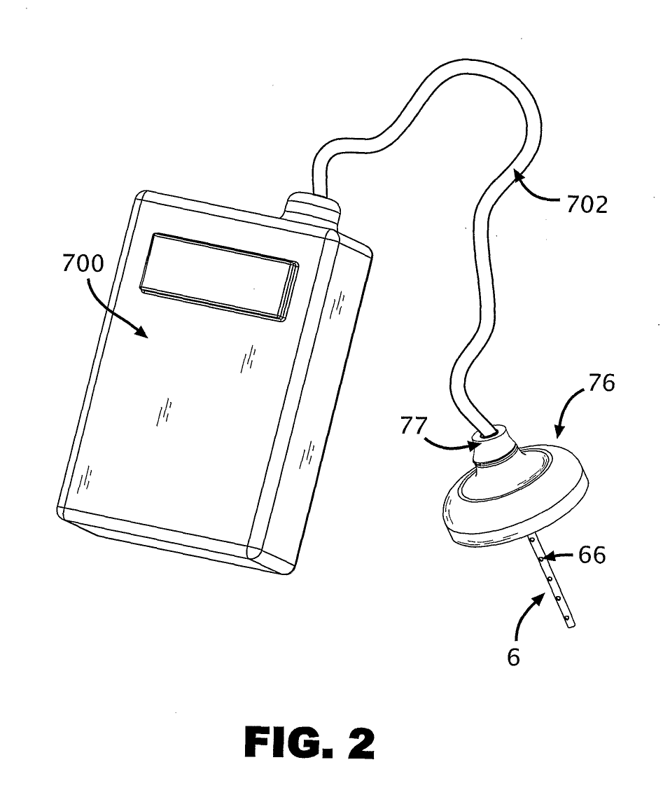

FIG. 2 illustrates an infusion device according to some embodiments that includes a pump 700. Therapeutic fluid can be delivered through an “infusion set”, which may include a tube 702, a port 76 attachable to the patient's skin (e.g., the port can be adhered to the skin via an adhesive layer), a connector 77, and a sprinkler cannula 6 (with holes 66). The connector 77 allows disconnection of the pump 700 and tube 702 from the port 76 upon patient's discretion.

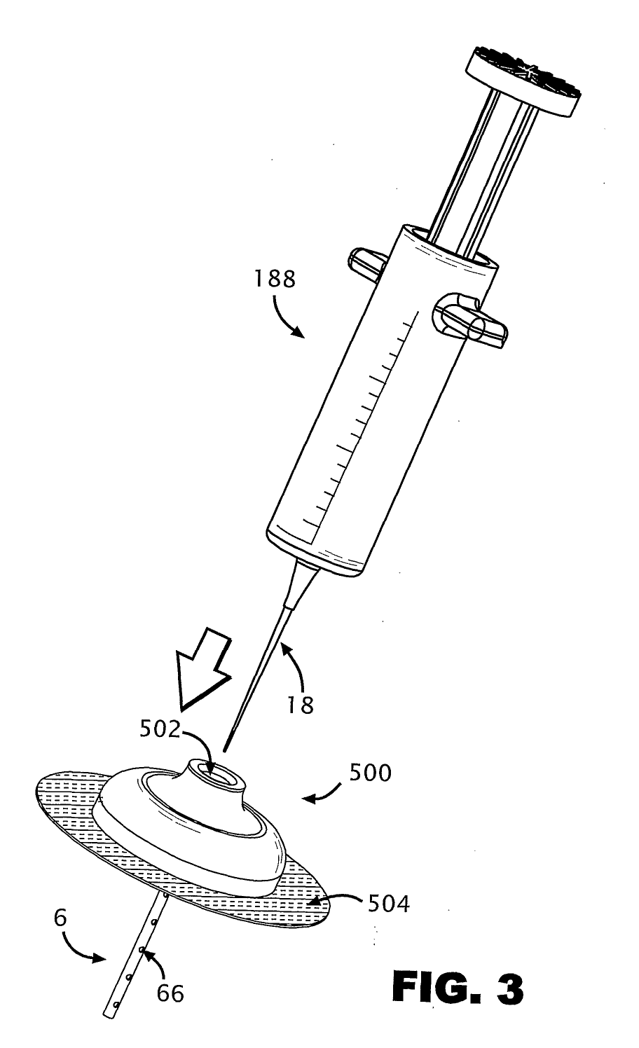

FIG. 3 illustrates a device according to some embodiments for drug delivery to the body. The device includes a sprinkler cannula 6 through which the drug is delivered and a skin adherable port 500. The port 500 can be adhered to the body via an adhesive tape 504, for example, and is connected to the sprinkler cannula 6 having holes 66. A self-sealable rubber septum 502 can be provided which allows for repeated piercing by a needle 18, for example, for establishing fluid communication between a syringe 188 (or other drug delivery device) and the sprinkler cannula 6. In some embodiments, the drug can be insulin administered to Type 1 or Type 2 diabetic patients.

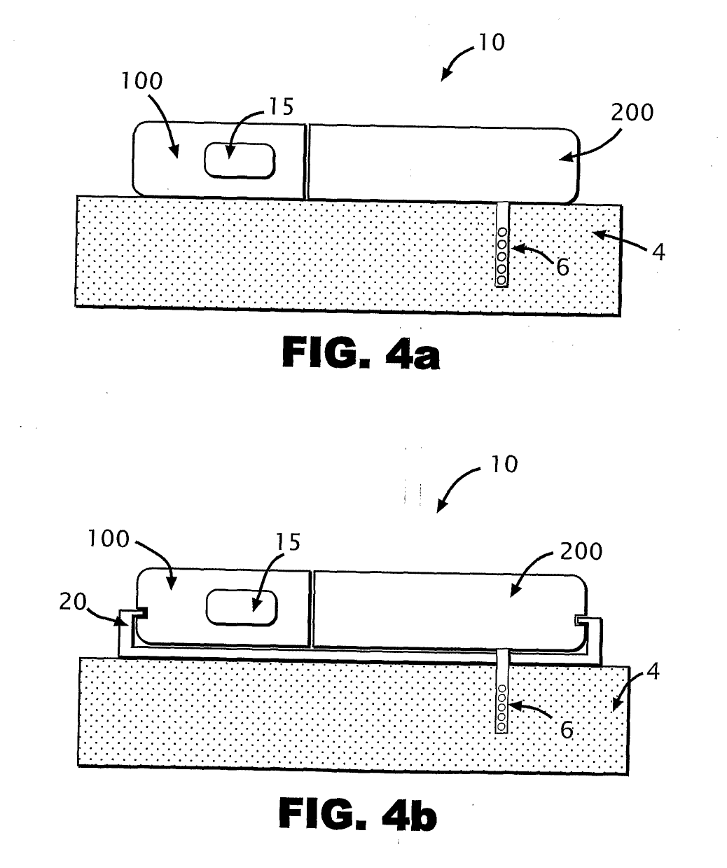

FIGS. 4a-b illustrate a device for drug delivery. The device includes a skin adherable dispensing patch 10 (also referred-to as “patch” or “dispensing unit”) and a cannula 6 with holes 66. FIG. 4a illustrates a cross sectional view of the dispensing patch 10 that is adhered to the patient's skin. The patch 10 can be composed of two parts, which may be, according to some embodiments, a reusable part 100 and a disposable part 200. In some embodiments, the dispensing patch 10 can be remotely controlled by a remote control or controlled directly using switches/buttons 15 (hereinafter “operating switches”) located on the reusable part 100 of the dispensing patch 10. In some embodiments, the remote control can include a dedicated remote control, a cellular phone, a PC, a laptop, a Personal Digital Assistant, a watch, a medial player (e.g., iPod), a smart phone (e.g., iPhone), and the like. In some embodiments, the switches 15 can include buttons, keys, a keypad, a touch sensitive user interface, a voice commander and the like. A sprinkler cannula 6 can be included which emerges from the disposable part 200, and can be in fluid communication with a fluid reservoir that is contained within the disposable part. FIG. 4b illustrates a cross sectional view of a dispensing patch 10 that can be connected to a skin adherable cradle 20. The dispensing patch 10 can be disconnected and reconnected from and to the cradle 20 upon patient's discretion.

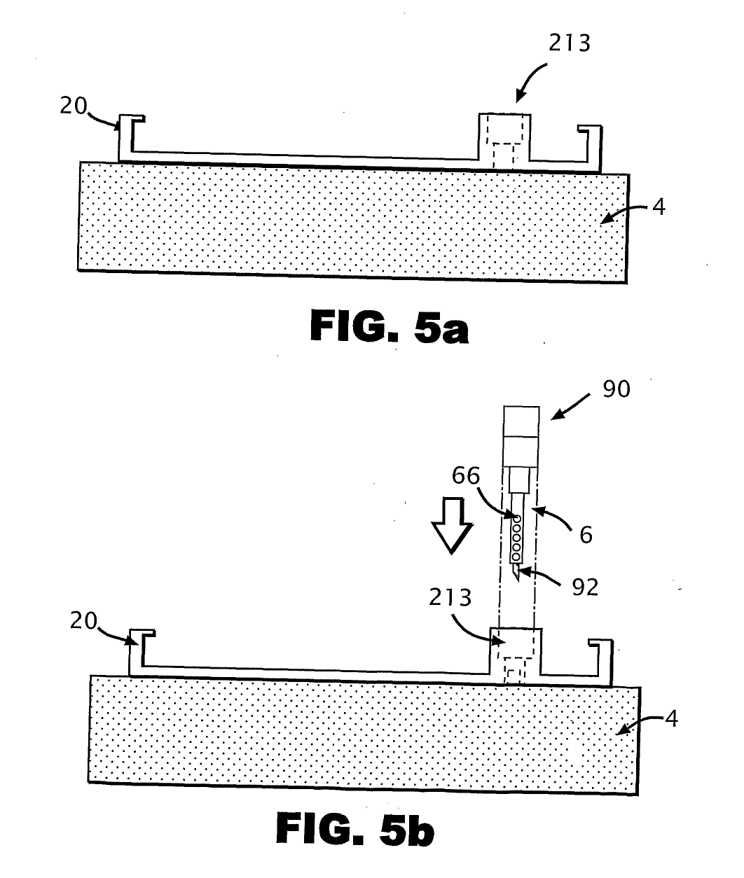

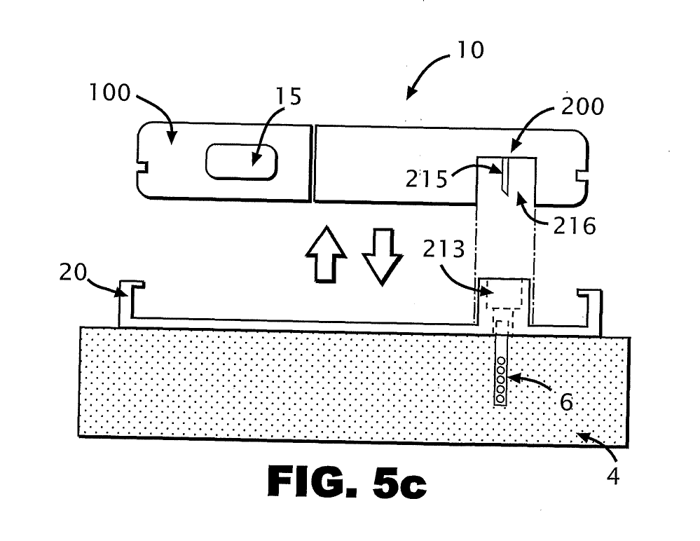

FIGS. 5a-c illustrate the insertion of sprinkler cannula 6 through a passageway 213 of skin adherable cradle 20 and into subcutaneous tissue 4. FIG. 5a illustrates a cross sectional view of the cradle 20 adhered to the skin, where the cradle 20 includes a protrusion which includes the passageway 213. FIG. 5b illustrates the insertion of a cannula cartridge 90 through the cradle passageway 213, where the cannula cartridge 90 can be composed of at least a penetrating member 92 (e.g., a needle) and sprinkler cannula 6 that includes holes 66. After inserting the cannula into the body, a cannula hub may be rigidly connected to the cradle 20 and the penetrating member 92 may then be retracted leaving the soft sprinkler cannula 6 placed within the subcutaneous tissue 4. FIG. 5c illustrates the connection of the dispensing patch 10 to the cradle 20. The patch 10 can be comprised of a first portion (e.g., a reusable part 100) having operating switches/buttons 15 according to some embodiments of the present disclosure, and a second portion (e.g., a disposable part 200). A recess 216 at the bottom of the patch may include a connecting lumen 215 which upon connection of the patch 10 to the cradle 20, the recess 216 receives the cradle protrusion and the connecting lumen 215 enables fluid communication between the reservoir, located in the second portion, and the sprinkler cannula 6. The patch 10 can be disconnected, and reconnected from and to the cradle 20 upon patient's discretion.

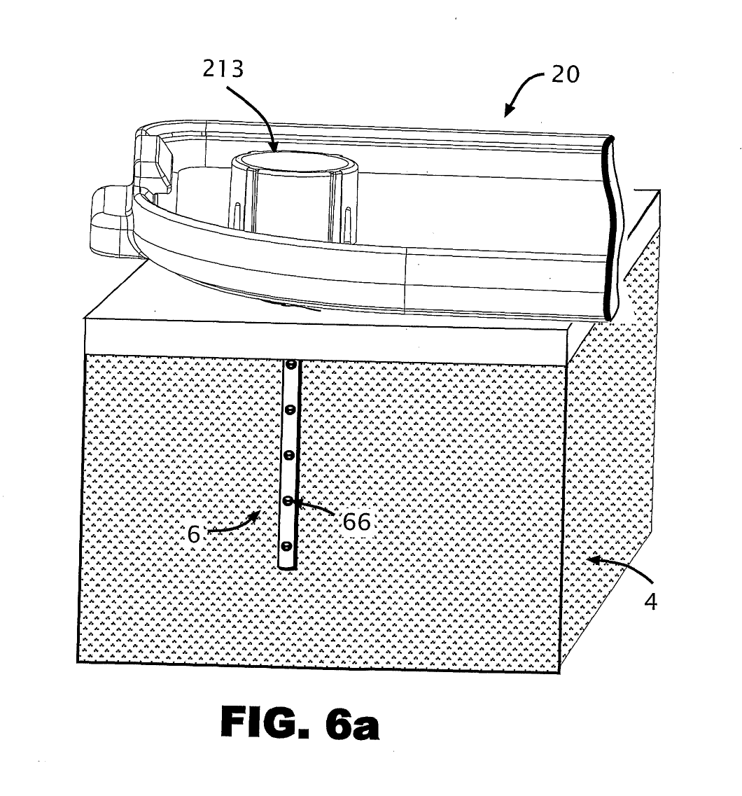

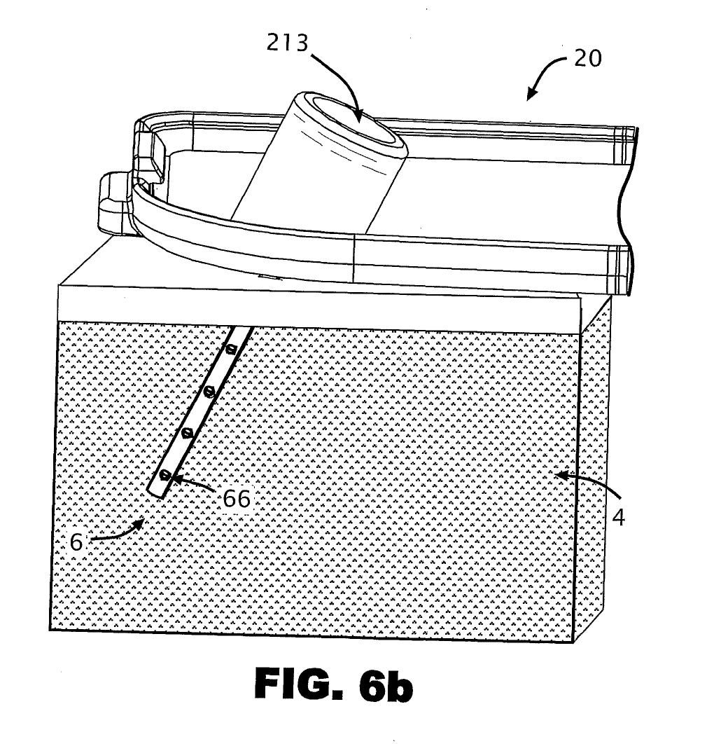

FIGS. 6a-b illustrate a portion of the cradle 20 according to some embodiments which includes a sprinkler cannula 6 with holes 66. The cradle includes a passageway 213 through which the sprinkler cannula 6 can be inserted into the subcutaneous tissue 4, either perpendicularly with respect to the skin surface (see FIG. 6a), or at another angle (see FIG. 6b) e.g., 30 degrees. In some embodiments, the passageway 213 can be tilted in various angles at patient's discretion.

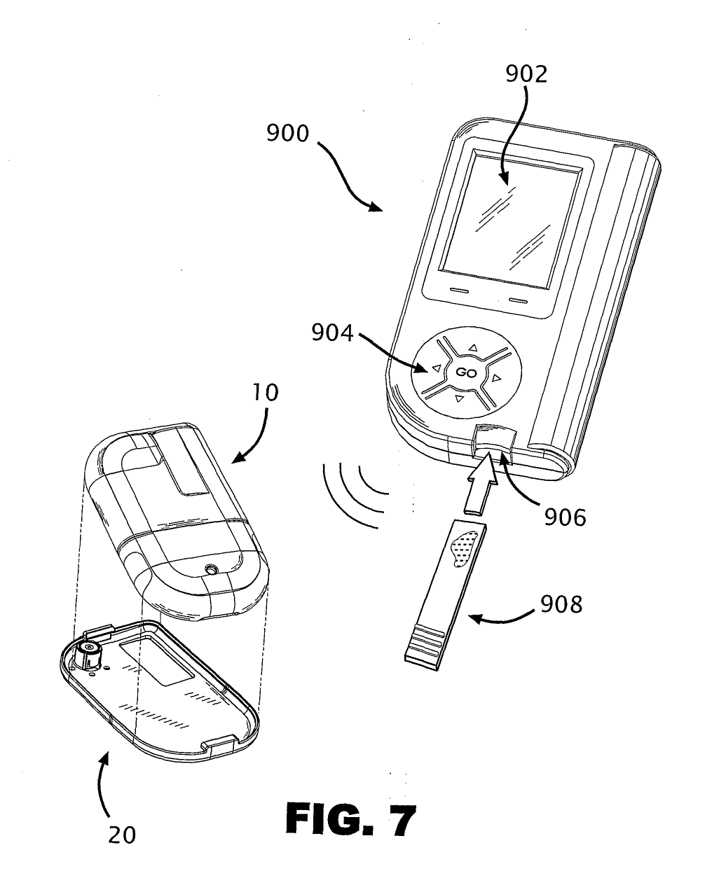

FIG. 7 illustrate a device (or system) according to some embodiments which comprises at least 3 units: (i) a dispensing patch 10, a skin adherable cradle 20 and a remote control 900. The patch 10 can be disconnected and reconnected from and to the cradle 20. The connecting lumen of the patch 10 enables fluid communication between the patch and the subcutaneously insertable sprinkler cannula that is preferably rigidly connected to the cradle. Fluid delivery can be remotely controlled by the remote control or by switches located on the patch 10.

In some embodiments, and as previously noted, the patch 10 can employ a pumping mechanism which includes a syringe with a propelling plunger. In some embodiments, the pumping mechanism can include a peristaltic mechanism having a tube, a magnetic mechanism or any other pumping mechanism known to one skilled in the art. The patch can further include a reservoir to retain the therapeutic fluid and an outlet port to enable fluid exit. In some embodiments, the patch can comprise a single part including a reservoir, one or more batteries, electronics, and driving mechanism (e.g., motor, gear) within a single housing. In some embodiments, the patch can comprise two-parts:
a. a reusable part (“RP”)—which can include a motor, gear(s), electronics (e.g., a processor or controller, a memory, a transceiver, a printed circuit board), and other relatively expensive components (e.g., sensors); and
b. a disposable part (“DP”)—which can include an outlet port, a reservoir, a slidable plunger, a drive screw, and a nut. The DP may contain the one or more batteries which supply power for patch operation.

In some embodiments, the RP can include at least a portion of a pump, and the DP may include another portion of the pump. Upon connection of the RP and the DP, the patch (e.g., therapeutic fluid delivery) is enabled.

In some embodiments, the one or more batteries can be located in the RP, in the DP, or shared between the two parts. In some embodiments, the one or more batteries can be rechargeable.

In some embodiments, the reservoir includes a flat profile (e.g., oval, ellipse, four arches) maintaining a relatively thin RP configuration. Each one of the RP and DP may include a housing. In some embodiments, the housing may include a shell or pocket and an “insert” (e.g., configured as a chassis). Accordingly, upon connection of the RP to the DP, the two housings and inserts are coupled.

In some embodiments, the cradle 20 is configured as a flat sheet (preferably rigid) with an adhesive layer facing the skin. The cradle is also provided with a passageway to receive a subcutaneously insertable cannula (e.g., sprinkler cannula) and may be provided with snaps, clasps, and/or latches to secure the cannula and patch (for example).

In some embodiments, the RC is configured as a handheld device for programming fluid flows, controlling the patch, data acquisition, and for providing indications to the user (e.g., via a display, speaker, vibration mechanism). As illustrated in FIG. 7, the RC 900 can include a screen 902, a keypad 904, and a blood glucose monitor. A test strip 908 can be received within a recess 906 so that glucose concentration can be evaluated and presented on the screen 902. In some embodiments, fluid delivery can be carried out based on the blood glucose concentration measured by the blood glucose monitor (e.g., in an open loop mode, semi-open loop mode or closed loop mode).

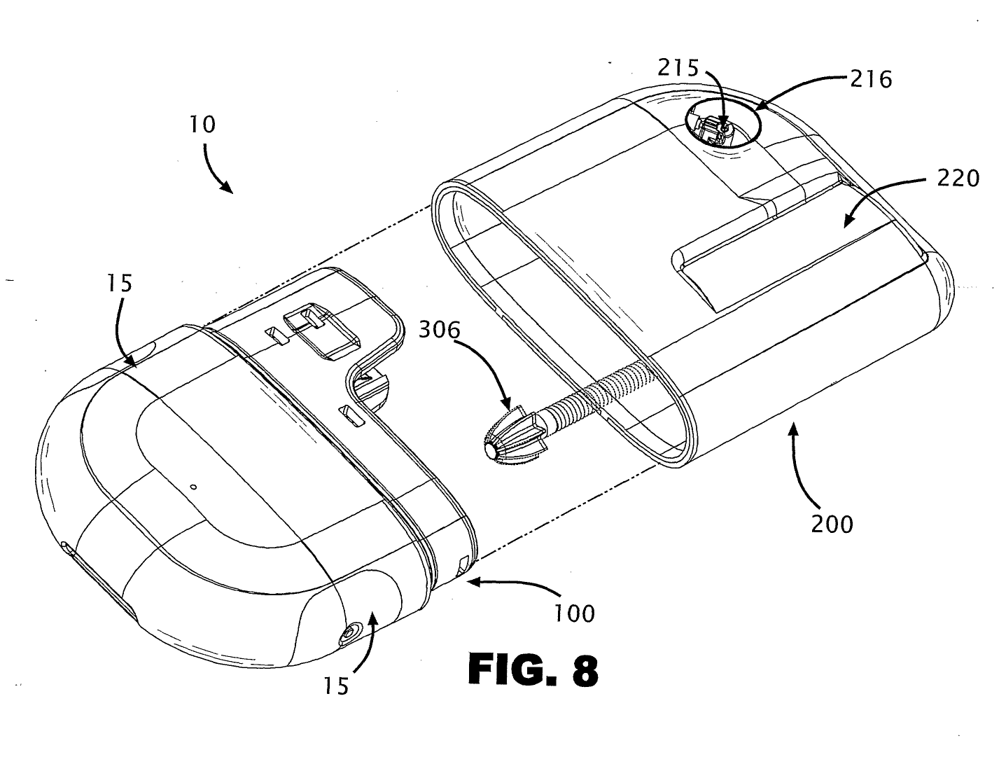

FIG. 8 illustrates an example of a two-part patch 10 that is comprised at least of a reusable part 100 and a disposable part 200. The reusable part 100 includes operating switches/buttons 15 and the disposable part 200 includes a reservoir 220 and an outlet port 216. Upon connection of the RP 100 and DP 200, the drive screw 306 can engage with a gear located in the RP to allow linear displacement of a plunger within the reservoir. The outlet port 216 includes connecting lumen 215 to enable fluid communication between the reservoir 220 and the sprinkler cannula (when connecting the patch to a cradle). A detailed description of this patch is disclosed in co-owned International Patent Application No. PCT/IL09/000,388 (published as WO2009/125398), claiming priority to U.S. Provisional Patent Application No. 61/123,509, entitled “Systems, devices and methods for fluid delivery”, filed on Apr. 9, 2008, the disclosures of which are incorporated herein by reference in their entireties.

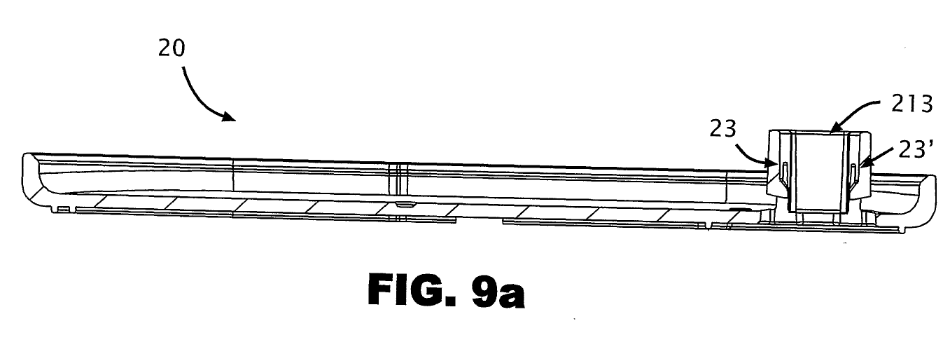

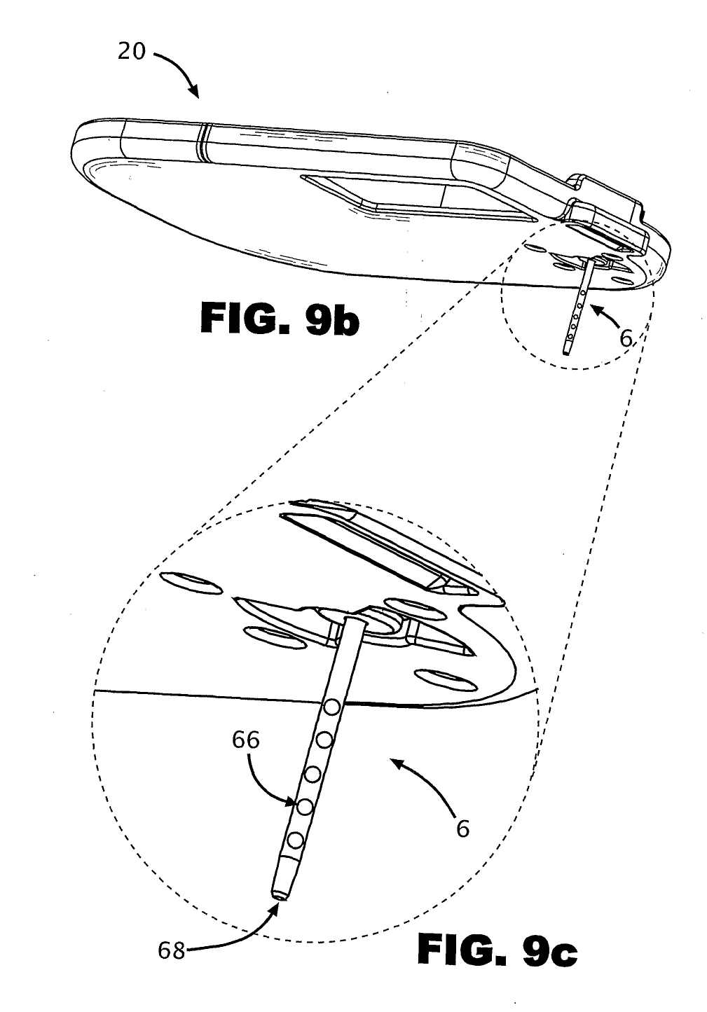

FIGS. 9a-c illustrate a spatial view of a cradle 20 before (see FIG. 9a) and after (see FIGS. 9b-c) attachment to a sprinkler cannula 6, according to some embodiments of the disclosure. FIG. 9a illustrates cradle 20 which may comprise a protrusion provided with a passageway 213. Two snaps/latches 23 and 23′, for example, provide connection (preferably rigid) of the sprinkler cannula 6 to the cradle 20 after insertion of the cannula 6 through the passageway 213. FIGS. 9b and 9c illustrate the sprinkler cannula 6 connected to cradle 20. The sprinkler cannula 6 may comprise a hole/opening at its tip 68 and a plurality of holes 66 along its longitudinal axis.

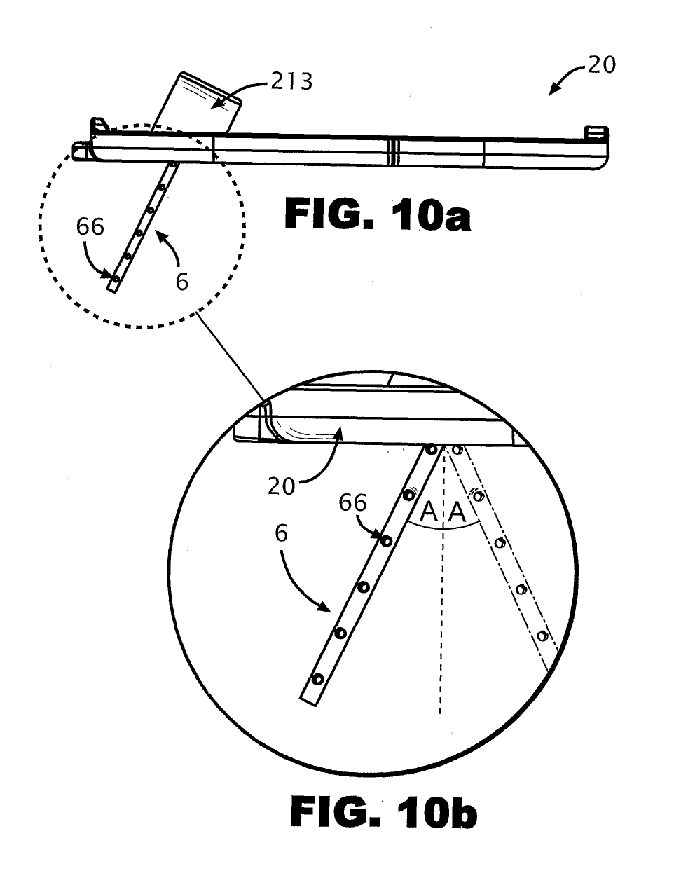

FIGS. 10a-b illustrate a cross sectional view of the sprinkler cannula 6 connected to cradle 20 at an angle “A”, provided with holes 66 is inserted through the passageway 213.

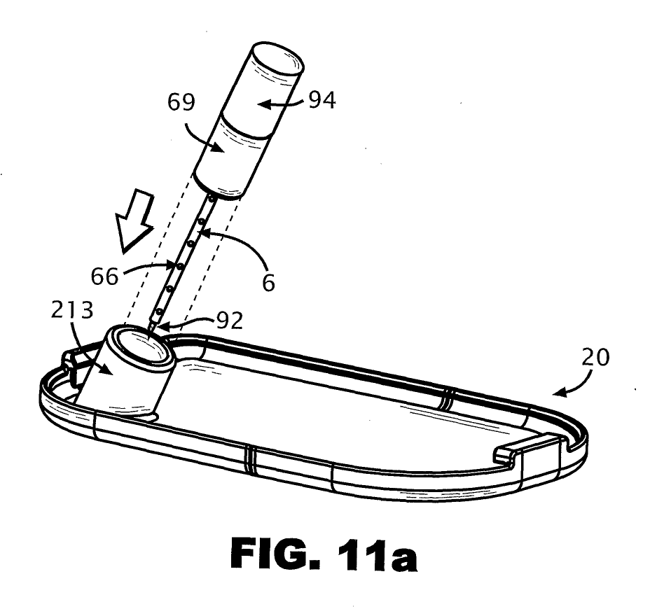

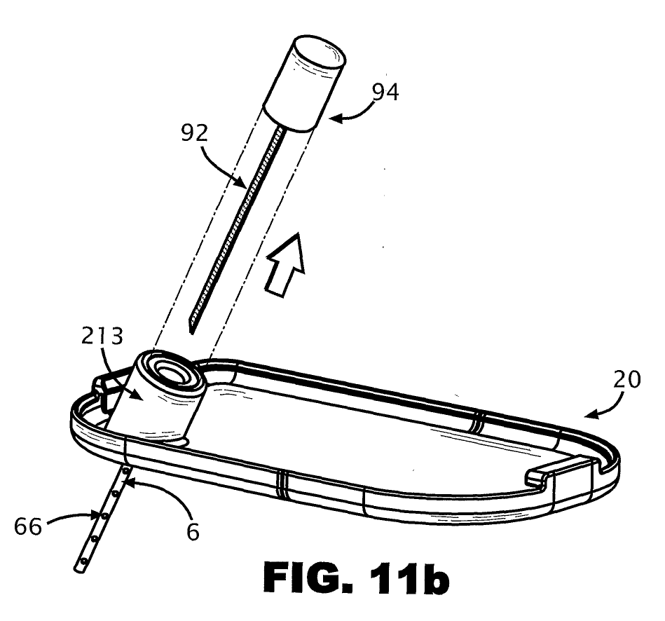

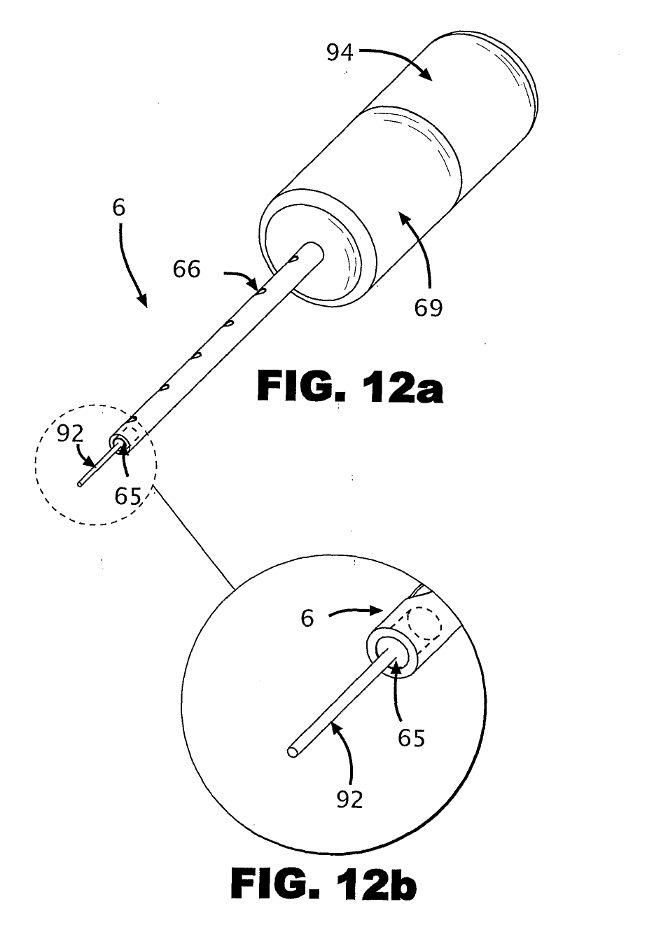

FIGS. 11a-b illustrate a tilted insertion of the sprinkler cannula 6 through the passageway 213 and into the subcutaneous tissue. A cannula cartridge 90 includes a sprinkler cannula 6, a penetrating member 92 with a sharp tip and a cap 94, and a cannula hub 69. After insertion (see FIG. 11b), the cannula hub 69 is preferably rigidly connected to the cradle 20 and the penetrating member 92 (including the cap 90) is retracted.

FIGS. 12a-b illustrate sprinkler cannula 6 provided with a self sealable seal 65 according to some embodiments. The cannula cartridge 90 preferably includes (before insertion) sprinkler cannula 6, a penetrating member 92 with a cap 94, and a cannula hub 69. According to some embodiments, the sprinkler cannula seal 65 seals the cannula tip after retraction of the penetrating member 92. In some embodiments, the seal 65 can be made of rubber that can be pierced by the penetrating member while being sealed after retraction of the penetrating member. The self sealable seal 65 can be used when drug delivery from the cannula tip is unnecessary or should be avoided while maintaining drug delivery through the sprinkler cannula holes 66.

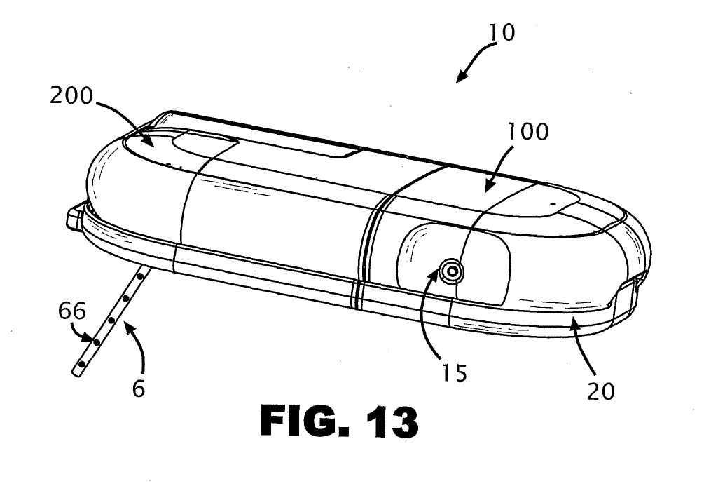

FIG. 13 illustrate a dispensing patch 10 connected to a cradle 20 according to some embodiments. The patch 10 can dispense fluid through the sprinkler cannula 6 into the body of a patient, where the patch 10 includes reusable part 100 and disposable part 200 and can be operated by operating switches/buttons 15.

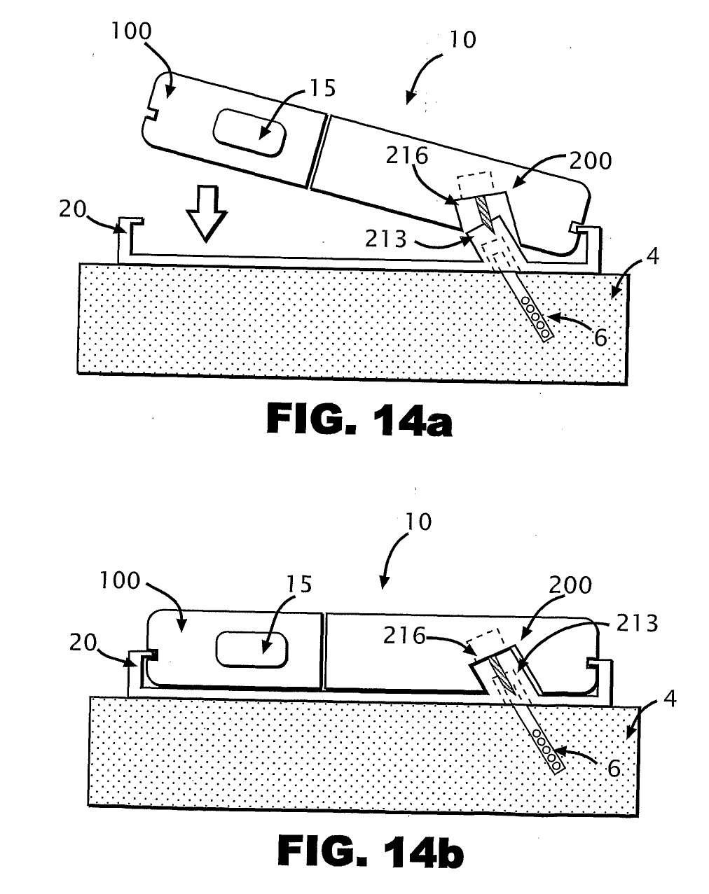

FIGS. 14a-b illustrate connection of dispensing patch 10 to skin adherable cradle 20 and skewed/tilted sprinkler cannula 6. FIG. 14a illustrates patch 10 which may include reusable part 100 and disposable part 200. The reusable part may include switches/buttons 15 (which may be used for operation). The disposable part may also include an outlet port 216 which can be configured as a recess in the bottom side of the patch 10 and may include a connecting lumen 215. The cradle 20 is preferably adherable to the skin and the sprinkler cannula 6 is inserted within the subcutaneous tissue 4 in an angled position. In some embodiments, the sprinkler cannula 6 can be placed, at least in part, in the cutaneous tissue. The outlet port 216 may be fitted with passageway 213 (e.g., within). The bold arrow indicates the direction of patch 10 displacement during its connection to the cradle 20. To disconnect the patch, it is displaced in the opposite direction. FIG. 14b illustrates the two-part patch 10 connected to cradle 20 where connecting lumen 215 pierces the septum of the cannula hub to establish fluid communication between the reservoir, located in the disposable part of the patch, and the sprinkler cannula 6.

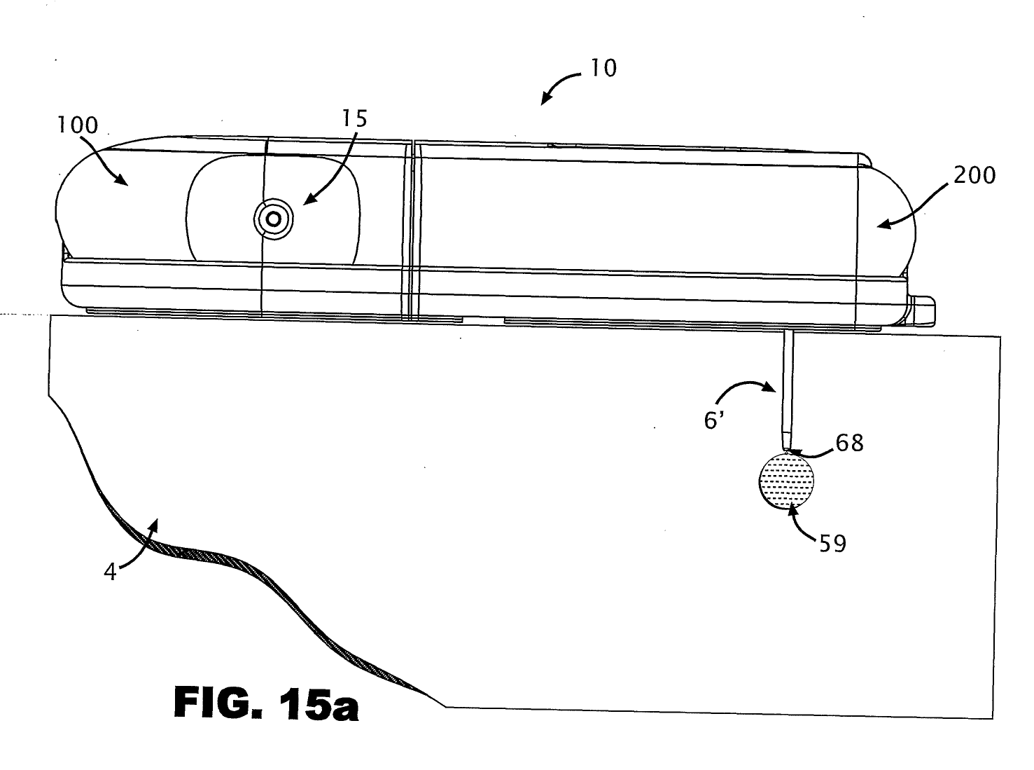

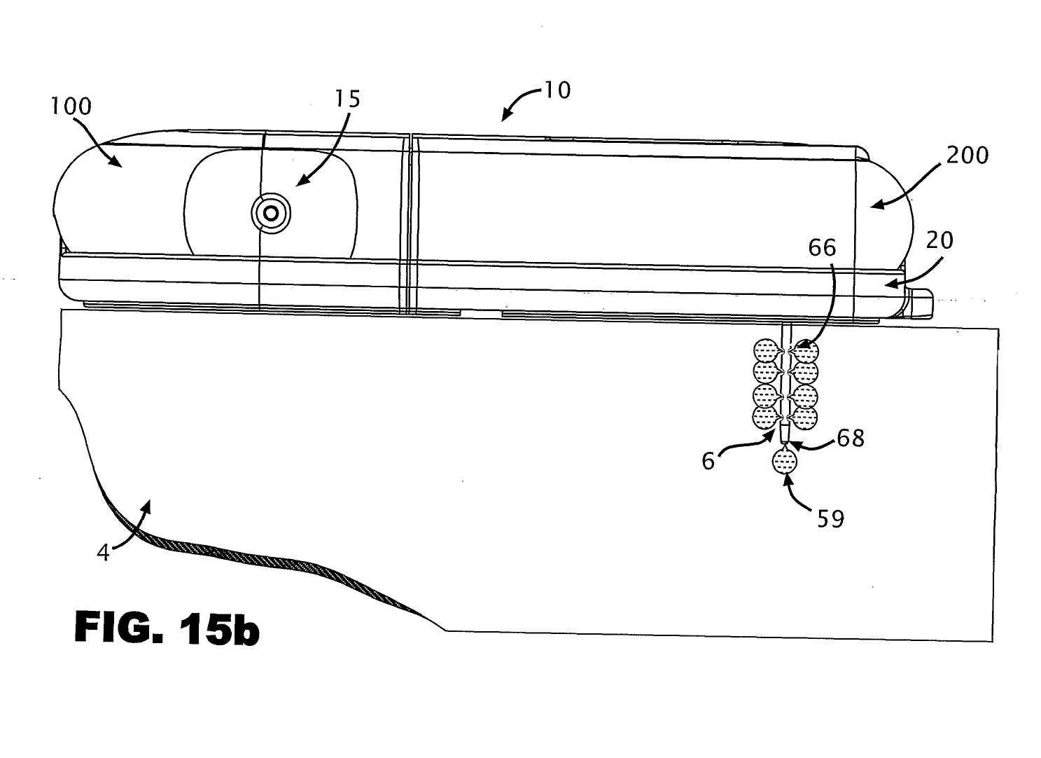

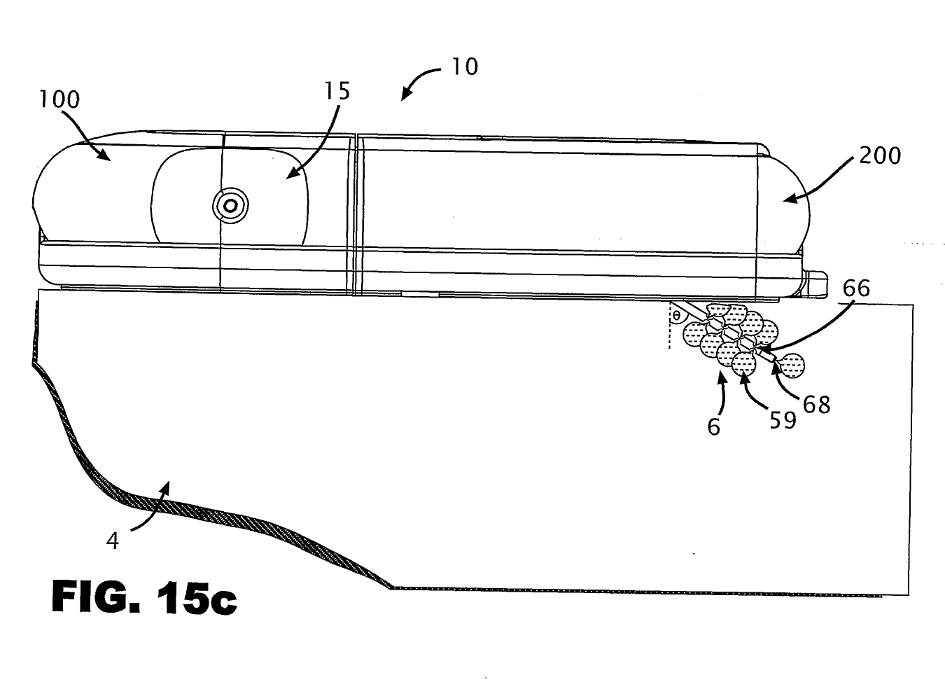

FIG. 15a illustrates a cross sectional view of a dispensing patch 10 comprised of two parts (100, 200) according to some embodiments, which is provided with a conventional cannula 6′ known in the art. The cannula 6′ is provided with a single fluid exit at the cannula tip 68. As fluid is dispensed through the cannula 6′, a depot 59 is formed at the cannula tip 68 and subsequently the fluid absorption process begins. In some embodiments, the absorption process may include diffusion of the drug (e.g., insulin) molecules in accordance with Fick's laws. Thus, the diffusion rate may be proportional to the effective diffusion area. For simplification, this principle can be illustrated in the following numerical example where a single depot of 10 U insulin with a concentration of 100 U/ml (V=100 mm³) has a surface area of about 104 mm² (under the approximation of a spherical depot). FIG. 15b illustrates the dispensing patch 10 that delivers fluid into the body through sprinkler cannula 6 that is located within the subcutaneous tissue 4. The fluid is delivered through the cannula tip 68 and through the plurality of holes 66 located along the longitudinal axis of the cannula 6 forms depot 59 at the cannula tip 68 and a plurality of depots at the holes 66 along the cannula side walls. The same volume of the depot of 10 U (100 mm³) described above, can now be divided, for example, into 10 depots (n=10) of 1 U each (V=10×10 mm³=100 mm³) and the total diffusion area of these 10 depots is about 224 mm² (i.e., more than twice of a single depot's). FIG. 15c illustrates sprinkler cannula 6 inserted within the subcutaneous tissue 4 in an angled/tilted direction. The drug depots 59 emerge from the sprinkler cannula holes 66 and from the cannula tip 68.

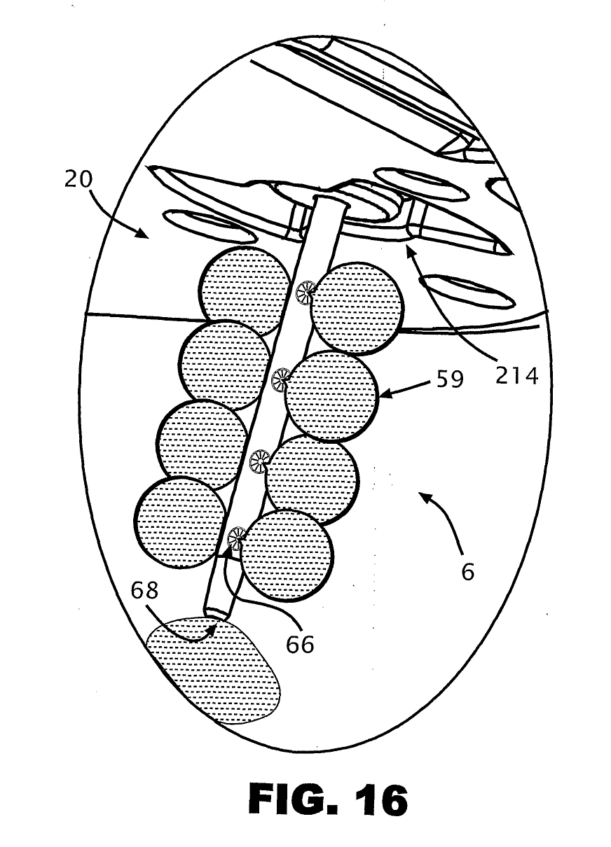

FIG. 16 illustrates sprinkler cannula 6 that includes holes 66 having unidirectional valves. The sprinkler cannula 6 is connected to cradle 20, emerging from a cradle opening 214. Drug depots 59 can be formed at the cannula side walls and at the cannula tip 68.

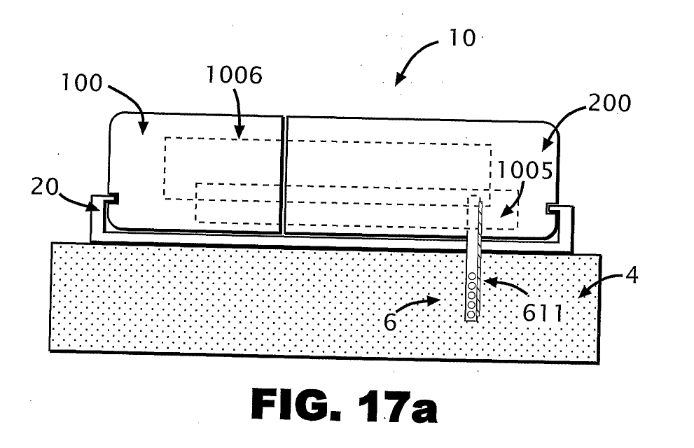

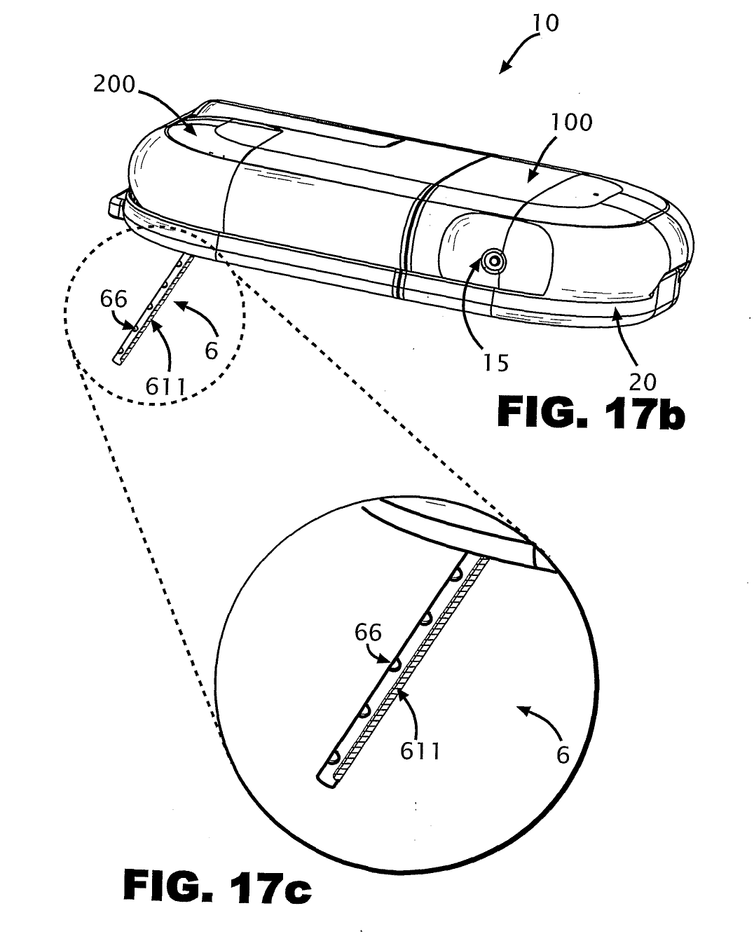

FIGS. 17a-c illustrate skin securable patch 10 that comprises dispensing apparatus 1005 for drug (e.g., insulin) delivery and sensing apparatus 1006 for continuously sensing of analytes (e.g. glucose) within the body. In some embodiments, insulin can be dispensed in correspondence with glucose readings forming a closed loop system. FIG. 17a illustrates a cross sectional view of the patch 10 that is comprises at least of a reusable part 100 and a disposable part 200. The patch 10 is connected to skin adherable cradle 20. The dispensing and sensing apparatuses (1005 and 1006 respectively) may be connected to a tip that is inserted into the subcutaneous tissue 4, where the tip may comprise a sprinkler cannula 6 for insulin dispensing and a sensing element 611 (also referred-to as “sensor”) for glucose sensing. FIG. 17b and FIG. 17c illustrate normal and magnified spatial views patch and tip.

Any and all references to publications or other documents, including but not limited to patents, patent applications, articles, webpages, books, etc presented and referenced in this specification are hereby incorporated by reference herein in their entireties. Although particular embodiments have been disclosed herein in detail, this has been done by way of example for purposes of illustration only, and is not intended to be limiting with respect to the scope of the appended claims, which follow.

It will thus be seen that many of the embodiments of the present disclosure attain objects made apparent from the preceding description. Since certain changes may be made to the inventions and corresponding embodiments disclosed herein without departing from the spirit and scope thereof, it is intended that all matter contained in the above description or shown in the accompanying drawings be interpreted as illustrative and exemplary, and thus, not limiting. Practitioners of the art will realize that the method, device and system configurations depicted and described herein are examples of multiple possible configurations that fall within the scope of the current disclosure. Any and all modifications of the embodiments disclosed herein are intended to be within the scope of claims appended hereto, as well as other claims which may be subsequently included in this or subsequent related filing.

1.-36. (canceled)

37. A drug delivery device for dispensing a drug into a tissue of a user/patient, the device comprising:
a reservoir for retaining a drug;
a cannula insertable into a tissue of a user/patient;
a pump for dispensing the drug from the reservoir into the tissue via the cannula, wherein:
the cannula comprises a soft tube having a plurality of apertures, the plurality of apertures being configured for delivering the drug into the tissue.

38. The device of claim 37, wherein the tube comprises a flexible tube.

39. The device of claim 37, wherein the apertures are disposed at one of around and along the tube.

40. The device of claim 37, further comprising a plurality of tubes.

41. The device of claim 37, wherein the plurality of apertures causes an increase in absorption rate of the drug in the tissue.

42. The device of claim 37, wherein the plurality of apertures are adapted to form a plurality of depots for the drug.

43. The device of claim 42, wherein the plurality of depots further correspond to an effective diffusion area substantially larger than an effective diffusion area formed from a single aperture forming a single depot.

44. The device of claim 37, wherein the tissue comprises a subcutaneous tissue and/or an intradermal tissue.

45. The device of claim 37, wherein at least one of the plurality of apertures is located in cutaneous tissue.

46. The device of claim 37, wherein:
the cannula is provided with a penetrating member having a sharp tip, the penetrating member is longitudinally traversable through the cannula, and after cannula insertion into the tissue, the penetrating member is retracted and the cannula is retained within the tissue.

47. The device of claim 37, wherein the pump is contained within at least one housing, the at least one housing is configured for repeated connection and disconnection to and from a skin adherable cradle, wherein the skin adherable cradle is provided with a passageway, and the cannula is insertable into the tissue through the passageway and is connected to the cradle after insertion.

48. The device of claim 37, wherein a proximal end of the soft tube is provided with a connector for establishing fluid communication between the reservoir and the cannula.

49. The device of claim 48, wherein the connector is fitted with a self-sealable septum, and wherein the self-sealable septum is configured for repeated piercing by a needle.

50. The device of claim 37, wherein the cannula and/or tube is made of a polymer.

51. The device of claim 37, wherein the plurality of apertures are configured with substantially similar dimensions.

52. The device of claim 37, wherein at least one aperture of the plurality of apertures is configured with one or more dimensions different from another aperture of the plurality of apertures.

53. The device of claim 37, wherein the cannula comprises a tip, and wherein the tip is provided with an opening.

54. The device of claim 53, wherein said opening is further adapted with a self-sealable septum.

55. The device of claim 37, wherein one or more of the plurality of apertures include at least one unidirectional valve.

56. The device of claim 37, wherein the cannula is configured for insertion into the skin of the user substantially perpendicular with respect to the skin surface.

57. The device of claim 37, wherein the cannula is configured for insertion into the skin of the user at an angle with respect to the skin surface.

58. The device of claim 37, wherein the cannula is configured for connection with a sensor, and wherein said sensor is capable of sensing an analyte concentration.

59. The device of claim 1, wherein the drug comprises insulin.

60. A cannula for dispensing of a drug into a tissue of a user/patient, the cannula comprising:
an elongated tube having a plurality of apertures disposed at least one of around and along the elongated tube, the plurality of apertures configured for delivering the drug into the tissue;
a connector provided on a proximal end of the tube for establishing fluid communication between a fluid delivery device and the cannula;
wherein the cannula is insertable into the tissue by using a rigid penetrating member having a sharp tip and,
wherein the plurality of apertures causes an increase in an absorption rate of the fluid in the body of the user/patient.

61. The cannula of claim 60, wherein the tube comprises a flexible tube.

62. A method for increasing the absorption rate of a therapeutic fluid in the body of a user/patient comprising:
providing a cannula for dispensing of the therapeutic fluid into a tissue of a user/patient, the cannula comprising:
a soft tube having a plurality of apertures, the plurality of apertures being configured for delivering the fluid to the tissue;
a connector provided on a proximal end of the soft tube for establishing fluid communication between a fluid delivery device and the cannula;
inserting the cannula into a tissue of the user/patient via a rigid penetrating member having a sharp tip;
and
dispensing the therapeutic fluid through the cannula, wherein the therapeutic fluid flows through the plurality of apertures and into the tissue of the user/patient resulting in an increase in the absorption rate of the therapeutic fluid in the body of the user/patient.
